(19) United States
(12) Patent Application Publication
(10) Pub. No.: US 2026/0258411 A1
(43) Pub. Date: Sep. 3, 2026

(54) CATALYTIC RNA FOR THE INDUCTION OF PROTEIN EXPRESSION AND/OR CODING A CONSORTIA POPULATION

(71) Applicant: William Marsh Rice University, Houston, TX (US)

(72) Inventors: James CHAPPELL, Houston, TX (US); Maria Claudia Villegas KCAM, Houston, TX (US); Kim Wai MO, Houston, TX (US); Jonathan SILBERG, Houston, TX (US)

(73) Assignee: William Marsh Rice University, Houston, TX (US)

(21) Appl. No.: 19/159,982

(22) PCT Filed: Feb. 27, 2024
(86) PCT No.: PCT/US2024/017481

§ 371 Date: Aug. 26, 2025

Related U.S. Application Data
(60) Provisional application No. 63/487,372, filed on Feb. 28, 2023.

(51) Int. Cl. C12N 15/113

(52) U.S. Cl. CPC C12N 15/113; C12N 2310/12; C12N 2310/20

(57) ABSTRACT

The present disclosure is directed to self-cleaving ribozyme constructs designed to target specific organisms and introduce exogenous sequences—both coding and non-coding-into expressed RNAs of the target host cell.

### PRIORITY CLAIM

This application claims benefit of priority to U.S. Provisional Application Ser. No. 63/487,372, filed Feb. 28, 2023, the entire contents of which are hereby incorporated by reference.

### STATEMENT REGARDING FEDERALLY SPONSORED RESEARCH

This invention was made with government support under Grant Nos. 2128370, 2227526, and 2237512 awarded by the National Science Foundation and Grant No. 2021-33522-35356 awarded by the United States Department of Agriculture/National Institute of Food and Agriculture. The government has certain rights in the invention.

### BACKGROUND

### 1. Field of the Disclosure

The present disclosure relates generally to the fields of medicine, infectious disease, and molecular biology. More particular, the disclosure relates to the use of catalytic RNAs in a variety of different applications, such as for introducing transgenes into multiple cells within a consortium and selectively expressing proteins and changing the phenotype within a targeted subset of the cells containing the transgene, such as bacterial cells, as well as biocontainment applications and consortia tracking, monitoring and screening.

### 2. Background

Ribozymes are ancient RNA molecules that evolved to perform catalytic reactions like enzymes, such as RNA splicing reactions in which covalent bonds are broken and reformed in RNA strands, i.e., ligation. These molecules have evolved to support a wide variety of reactions, such as RNA splicing, site-specific cleavage, control of gene expression, viral replication, and protein biosynthesis. A wide range of ribozymes have been discovered. The hammerhead ribozyme is probably the best-characterized family member and was the subject matter relating to the 1989 Nobel Prize in Chemistry. Because ribozymes are made of RNA, they are also capable of binding to other RNA using Watson-Crick base pairing. Ribozymes can also form protein-RNA hybrid complexes such as in the ribosome.

Ribozymes can be separated into two main categories: (1) self-cleaving and (2) self-splicing ribozymes (divided into group I and group II introns). The first splicing ribozyme to be characterized was the group I intron that is natively transcribed as an intervening sequence within the Tetrahymena thermophila 26S rRNA. Once expressed, the ribozyme removes itself from the transcript and splices together the flanking RNA sequences. The Tetrahymena thermophila ribozyme is 413 bp long and forms a complex secondary and tertiary structure (FIG. 1A). An overview of the reaction catalyzed by this ribozyme is shown in FIG. 1B. Specifically, this ribozyme catalyzes the formation of a phosphodiester bond between the 5′ splice site (5′SS) and 3′ splice site (3′SS).

Shortly after its discovery, it was recognized that the splicing ribozyme can be modified to perform a trans-splicing reaction (i.e., ligation of a 3′ exon onto a separate RNA strand). In this scenario, the ribozyme is attached to the 3′ exon and forms interactions to a separate RNA (called target RNA herein) leading to splicing. This functionality was quickly recognized for its therapeutic potential for two main application spaces:
(1) To repair defective transcripts: For example, to replace a mutated sequence with a correct sequence. This includes repairing model genes, endogenous myotonic dystrophy protein kinase transcripts, and sickle-globin transcripts (Phylactou et al., 1998; Lan et al., 1998; Sullenger & Cech, 1994). These first-generation designs generally performed poorly, in part due to the limited length of interactions between the target RNA and the ribozyme.
(2) To selectively kill a targeted diseased cell: For example, cancer cells can be killed with exquisite specificity by trans-splicing ribozymes that target sites on RNA expressed highly in cancer cells (such as hTert RNA) by splicing a ‘death gene’ onto the target RNA, e.g., diphtheria toxin A (Ayre et al., 1999) or herpes simplex virus thymidine kinase (HSV-tk), which converts the drug ganciclovir to a cytotoxin (Kwon et al., 2005). Other applications include splicing activity without affective cell fitness and selectively killing of related cells that are infected with viruses (Ayre et al., 1999; Carter et al., 2010; Ryu et al., 2003).
   The state of these technologies is unclear as most people use CRISPR-type systems for such gene editing these days. As such, there remains an untapped potential for the use of trans-splicing ribozymes, especially in achieving control across evolutionary-divergent species within a consortium of cells.

### SUMMARY

Thus, in accordance with the present disclosure, there is provided a ribonucleic acid (RNA) segment comprising a catalytically functional core from a trans-splicing catalytic RNA, a synthetic guide RNA (sgRNA), and an open reading frame (ORF) encoding a peptide or protein that encodes a protein or peptide of interest, wherein the sgRNA targets a RNA transcript such that a trans-splicing reaction by said catalytic RNA results in the introduction of said ORF into said RNA transcript such that a translational control signal of said RNA transcript is positioned in operable relation to said ORF. The said RNA segment may lack any translational control signals and/or lacks an origin of replication. The ORF may encode a detectable marker, a protein conferring growth/survival capability on said cell, a protein that generates overt information, a protein that produces a signal containing information, an antimicrobial, an anti-fungal agent, an anti-viral agent, anti-archaean agent or a toxin. The translational control signal may be a translation initiation signal and/or ribosome binding site. The catalytic RNA may be derived from Tetrahymena, such as a group I intron, or is synthetic.

The said sgRNA may (a) target a eukaryotic, mammalian, human, bacterial, fungal, archaean or viral transcript; (b) be fully or partially complementary to said RNA transcript; and/or (c) comprise all natural ribonucleotides, all non-natural ribonucleotides, or a mix of natural and non-natural ribonucleotides. The catalytically functional core may be about 200,300 or 400 ribonucleotides in length, is about 200-500, 300-500, or 400-500 ribonucleotides in length, is about 200-600, 300-600, or 400-600 ribonucleotides in length, is about 200-800, 300-800 or 400-800 ribonucleotides in length, or is about 200-100-, 300-1000 or 400-1000 ribonucleotides in length. The sgRNA may be about 25 to about 375 ribonucleotides in length. The RNA segment may further comprise a unique barcode or identifier that will be introduced into said RNA transcript after said trans-splicing reaction. The RNA transcript may be an engineered transcript, such as one comprising a non-hydrolyzable nucleic acid backbone, or a native transcript, such as a transcript arising from a low copy number gene.

Also provided is a nucleic acid vector encoding an RNA segment as described herein. The vector may be an RNA or DNA vectors, such as a viral vector, such as a baculovirus vector or a phage. The vector may be DNA vector, such as a non-viral vector, such as a plasmid (e.g., self-mobilizing or non-self-mobilizing). Another embodiment comprises a pharmaceutical formulation comprising an RNA segment or vector as described herein. A further embodiment comprises a cell comprising an RNA segment or vector as described herein.

In yet another embodiment, there is provided a method of expressing a protein or peptide in a cell comprising contacting said cell with an RNA segment or vector as described herein under conditions permitting uptake of said RNA segment or vector by said cell and trans-splicing of said RNA transcript by said catalytic RNA. The cell may be a bacterial cell, a fungal cell, a parasite cell, a mammalian cell, or a human cell. The RNA segment may be contacted with said cell as naked RNA. The RNA segment may be contacted with said cell packaged in a nanoparticle or liposome.

The RNA segment may encode a peptide or protein that is toxic to said cell, and said method results in the death of said cell. The cell may be part of a consortium of cells, such as wherein said consortium contains one or more organisms that are pathogenic to a plant, mammal or human. Contacting may result in transduction of, transformation of or conjugation with said cell. The ORF may encode a detectable marker and said method further comprises detecting expression of said detectable marker, such as quantitative detection. The trans-splicing may result in constitutive expression of said peptide or protein. The trans-splicing may permit conditional expression of said peptide or protein when other conditions are met.

The RNA segment may encode a peptide or protein that it confers a growth or survival advantage to said cell under all or selective environments. The cell may be part of a consortium of cells. The consortium may be beneficial to a plant, mammal or human. The consortium may express a protein of interest.

In yet additional embodiment, there are provided methods of biocontained supplementation comprising:
(a) providing an engineered organism that provides a benefit to a plant or microbe and comprises a ribonucleic acid (RNA) segment comprising a catalytically functional core from a trans-splicing catalytic RNA, a synthetic guide RNA (sgRNA), and an open reading frame (ORF) encoding a peptide or protein that encodes a protein or peptide that is toxic to said engineered organism, wherein the sgRNA targets an RNA transcript such that a trans-splicing reaction by said catalytic RNA results in the introduction of said ORF into said RNA transcript such that a translational control signal of said RNA transcript is positioned in operable relation to said ORF, and wherein the RNA segment is expressed conditionally when said engineered organism is outside a defined environment; and
(b) contacting said engineered organism with said plant or microbe; or
(a) providing an engineered organism that encodes a gene product that provides a benefit to a plant or microbe and comprises a ribonucleic acid (RNA) segment comprising a catalytically functional core from a trans-splicing catalytic RNA, a synthetic guide RNA (sgRNA), wherein the sgRNA targets an RNA transcript that encodes said gene such that a trans-splicing reaction by said catalytic RNA results in the disruption of the RNA transcript encoding said gene product that translation of said RNA transcript is disrupted, and wherein the RNA segment is expressed conditionally when said engineered organism is outside a defined environment; and
(b) contacting said engineered organism with said plant or microbe; or.
(a) providing an engineered organism that comprises a ribonucleic acid (RNA) segment comprising a catalytically functional core from a trans-splicing catalytic RNA, a synthetic guide RNA (sgRNA), and an open reading frame (ORF) encoding a peptide or protein that encodes a protein or peptide that confers a benefit on a plant or microbe, wherein the sgRNA targets an RNA transcript such that a trans-splicing reaction by said catalytic RNA results in the introduction of said ORF into said RNA transcript such that a translational control signal of said RNA transcript is positioned in operable relation to said ORF, and wherein the RNA segment is expressed conditionally when said engineered organism is inside a defined environment; and
(b) contacting said engineered organism with said plant or microbe.

In yet another embodiment, there is provided a method of labelling one or more cells a consortium comprising contact said consortium with an RNA segment or the vector as defined herein under conditions permitting uptake of said RNA segment or vector by said cell and trans-splicing of said RNA transcript by said catalytic RNA. The said consortium may comprise bacterial cells, fungal cells, parasite cells, mammalian cells, or human cells. The RNA segment may be contacted with said consortium as naked RNA or may be contacted with said consortium packaged in a nanoparticle or liposome. The RNA segment may encode a nucleic acid sequence that is not found in members of said consortium. The consortium may comprise mammal or human pathogens or may comprise cells beneficial to a mammal or human. The RNA segment may further encode (a) a peptide or protein that it confers a growth or survival advantage to cells of said consortium under specified conditions or (b) encodes a peptide or protein that it toxic to cells of said consortium. The consortium may express a protein of interest. The contacting may result in transduction of, transformation of or conjugation with said cell. The trans-splicing may result in constitutive expression of said peptide or protein. The trans-splicing may permit conditional expression of said peptide or protein when other conditions are met.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present disclosure. The disclosure may be better understood by reference to one or more of these drawings in combination with the detailed description of specific embodiments presented herein.

FIGS. 1A-B. Ribozyme structure and function. (FIG. 1A) Structure of the Tetrahymena splicing ribozyme. Labeled are the paired domains (e.g., P1, P2, P3 etc.), internal guide sequence (IGS), 5′ and 3′ splice sites, 5′ and 3′ exons, G-site, and wobble G. (FIG. 1B) Cartoon of the splicing reaction in which the ribozyme (black) joins the 5′ and 3′ exon (blue) together. In brief, an exogenous Guanosine Triphosphate (GTP) binds to the G-site of the ribozyme. The GTP then performs a nucleophilic attack on the phosphodiester bond at the 5′SS, leaving a free uracil at the 5′ exon. The exogenous GTP then leaves the G-site, allowing the 3′ wobble G of the ribozyme to bind there. The free 3′ hydroxyl on the 5′ exon then attacks the phosphodiester bond at the 3′ Splice Site between the wobble G and the start of the 3′ exon, breaking the bond at the 3′ Splice Site and forming a bond between the 5′ exon hydroxyl and the 3′ exon phosphate. This reaction forms the functional transcript, with the 5′ exon and the 3′ exon scarlessly ligated, and the ribozyme is excised from the transcript.

FIGS. 2A-B. Using cat-RNA to selectively visualize and kill pathobionts and pathogens. (FIG. 2A) When a plasmid encoding a cat-RNA (black) fused to an output gene (red) enters a cell, the fused transcript (Cat-RNA: Output) binds to a host-derived transcript (blue) and splices the output gene onto the host transcript. (inset) The synthetic RNA guide that directs host transcript binding only requires one uracil in the target RNA and reverse complementarity to the target. Splicing creates an output mRNA that can be translated. (FIG. 2B) Two example applications include using cat-RNA to switch on (top) the synthesis of visual reporters to enable selective detection or (bottom) the production of toxins to create selective antimicrobials.

FIGS. 3A-B. Cat-RNA Can Produce Protein Outputs From Cellular RNA. (FIG. 3A)

Schematic of the assay in which a fluorescent protein is produced by targeting Cat-RNA to a cellular RNA in E. coli cells. (FIG. 3B) Characterization of protein production by fluorescence measurements shows that Cat-RNA can produce GFP in cells when the target RNA is present. Data show the mean and standard deviation of n=3 biological replicates and asterisk (*) indicates p values of >0.001 as determined by a T test.

FIGS. 4A-C. Cat-RNA can produce toxic protein outputs to kill target cells. (FIG. 4A) Schematic of the assay in which a toxic protein is produced by targeting Cat-RNA to a cellular RNA in E. coli cells. (FIG. 4B) Plating assays to measure the colony-forming units (CFU) after cat-RNA expression is induced or not induced in cells for 6 hours. (FIG. 4C) Graph showing CFU data for non-induced and induced cat-RNA. Labels indicate different output genes that are posited to kill the host cell. Data shows mean and standard deviation of n=2 biological replicates.

FIGS. 5A-C. Rapid scoring of Cat-RNA activity using a growth selection. (FIG. 5A) Schematic of the assay in which an enzyme (methyl halide transferase, MHT) is produced by targeting Cat-RNA to a cellular RNA in E. coli cells. Cells containing the most active cat-RNA designs are able to grow faster compared to variants with less efficient cat-RNA designs. (FIG. 5B) Log enrichment data of ~50 cat-RNA variants targeting different locations on a cellular RNA (mRFP transcript). (FIG. 5C) Data showing log enrichment data is highly correlated with protein output, in this case, MHT which is quantified using GC-MS. The Cat-RNA protein output represents the MHT catalytic product, methyl halide.

FIGS. 6A-C. Diverse mRNA sequences can activate antibiotics. (FIG. 6A) Sensing modules of various sizes can mediate output activation, and thus, can be used to tune target specificity. (FIG. 6B) Within a single mRNA, many sequence motifs with a U can be used to trigger output production, although each differs in efficiency. In this case, the output is a visual reporter. (FIG. 6C) Across eight mRNA, many unique sequence motifs can trigger smart RNA outputs. The top five most active designs for each target mRNA are shown, although >10³ were tested.

FIGS. 7A-B. Tracking DNA delivery to microbiomes. (FIG. 7A) To track DNA delivery a method can be used that uses a universal RNA editor that binds to a conserved 16S rRNA motif (gray) found in all cells. When cells take up DNA coding for this RNA editor, barcodes (green) are added to 16S rRNA. Next generation sequencing is then used to identify who took up DNA. (FIG. 7B) Application of this tool in a wastewater microbiome identified ~200 species (tree), whose abundances varied (green), and 45% of which took up the DNA (blue). This universal editor is compatible with studying DNA delivery using transformation, transduction, and conjugation, and can be used to track delivery of Cat-RNA.

FIG. 8. The antibiotic challenge. (left) Chemical antibiotics frequently kill microbes non-specifically, which can cause health complications and drug resistance. (right) Smart RNA antibiotics interact with diverse species in the microbiome, but only kill pathogens containing unique mRNA cell signatures.

FIGS. 9A-D. Smart RNA antibiotics. (FIG. 9A) Like a trojan horse, smart RNA enters a microbiome and move silently from microbe to microbe. When they enter a pathogen, they bind to a pathogen mRNA which triggers them to synthesize a toxin. These therapeutics can: (FIG. 9B) be designed to be activated by diverse mRNA inputs, (FIG. 9C) be rapidly refactored for personalized medicine, and (FIG. 9D) selectively kill pathogens in complex microbiomes.

FIG. 10. Biocontainment. The inventors propose that environmental signals from different environments (e.g., human gut, soil, built, wastewater, and more) can be used to repress the production of smart RNA, such that if these microbes escape that environment, smart RNA production is activated and the microbes are killed via smart antibiotic production.

FIGS. 11A-B. Cat-RNA can specifically target a pathogenic gene and achieve strain-specific killing in the pathogen E. coli ST131. (FIG. 11A) A pathogen-specific Cat-RNA (killing activity measured by colony forming units in Logo scale) in E. coli ST131. The activation of the pathogen-specific Cat-RNA was controlled by a cumate inducible promoter (induced or not induced shown). No Cat-RNA is an empty plasmid control without Cat-RNA encoded within it that should not kill cells. ST131 Cat-RNA targets pathogenic-specific gene aacC3 and produces a toxin called RhsA. Data shows that when present (induced) the inventors see killing of the ST131 by the ST131 Cat-RNA design. When the Cat-RNA is not induced the inventors observe no killing compared to the No Cat-RNA control. (FIG. 11B) Pathogen-specific Cat-RNA in non-pathogenic E. coli BW25113 that lacks the target gene aacC 3. No Cat-RNA is an empty plasmid control without Cat-RNA encoded within it. In this experiment, the inventors see no killing of E. coli BW25113 compared to the no CAT-RNA control. Taken together these results suggest the inventors can selectively kill E. coli ST131 using Cat-RNA. FIG. 12. Delivery of Cat-RNA by P1 phagemids. In this experiment, phagemids were used to deliver different plasmids into E. coli cells. Specifically, a plasmid encoding a GFP gene (positive control in which no killing should be observed), a Cat-RNA that kills cells containing an RFP gene (CAT-RNA-RFP), and a Cat-RNA that kills cells containing an ampicillin resistance gene (Cat-RNA-AmpR). These cells were introduced into two populations of E. coli cells containing either the RFP (orange bars) or AmpR (blue bars) gene. When E. coli were selected for transduction (i.e., delivery of a plasmid) and the killing efficiency of the different Cat-RNAs measured by colony forming units (CFU), the inventors observed the following. As expected, no killing is observed when the GFP plasmid was delivered. When the Cat-RNA targeting RFP was delivered, the inventors observed specific killing of cells containing RFP but no killing of cells containing the AmpR gene. When the Cat-RNA targeting AmpR was delivered, the inventors observed specific killing of cells containing AmpR but not cells containing the RFP gene.

FIG. 13. Cat-RNA can activate the production of proteins using diverse endogenous genome-encoded mRNA in E. coli cells. In this experiment, the ability to produce a protein output using Cat-RNA targeting low-copy endogenous genes encoded on the E. coli genome was confirmed. This experiment uses a methyl halide transferase (MHT) output from Cat-RNA that produces CH₃Br gas which is measured by GC-MS. Additionally, CO₂ is measured as a proxy of cell growth and used to normalize the CH₃Br signals produced by each culture. E. coli cells were transformed with different plasmids encoding Cat-RNA designed to target different endogenous genes that include: aceE, tig, tufA, rimM, groL, hfq, sodA, atpA, cspC, groL, iscS, ompC, and yceD. A no guide RNA control was also included that should not produce the MHT output and the CH₃Br output. Two types of Cat-RNA design were tested called TTTPlex and compPlex that vary in a structural feature within the ribozyme. Compared to the negative control, the inventors observed strong CH₃Br production from the different designs targeting different genes, suggesting that Cat-RNA is able to produce the MHT output from different endogenous genome-encoded mRNA.

FIG. 14. Cat-RNA selectively activate the production of proteins using endogenous genome-encoded mRNA in E. coli cells. To confirm that Cat-RNA was able to selectively activate MHT production from the intended gene target, the inventors tested the activity of a series of Cat-RNAs targeting the endogenous gene OmpC in a wild-type E. coli (+OmpC) and an E. coli strain in which the OmpC gene had been knocked out (−OmpC). This experiment uses a methyl halide transferase (MHT) output from Cat-RNA that produces CH₃Br which is measured by GC-MS. E. coli cells were transformed with different plasmids encoding Cat-RNA designed and a no guide RNA control was also included that should not produce the MHT output. From these experiments, the inventors saw that for many designs they had a significant reduction in CH₃Br production in the absence of the gene target (OmpC), suggesting that the inventors are able to selectively activate protein production using endogenous genes.

### DESCRIPTION OF ILLUSTRATIVE EMBODIMENTS

Traditional chemical antibiotics kill both good (commensal) and bad (pathogenic) microbes, and thus, massively disrupt the microbial communities (microbiomes) that live in and on our body, which support health by synthesizing chemicals, regulating our immune system, and inhibiting pathogen growth. Unfortunately, the overuse and non-specific nature of antibiotics has led to an explosion of drug-resistant pathogens, nullifying our arsenal against infection. With antibiotic discovery rates diminishing and the cost of drug development skyrocketing, there is a threat of returning to the pre-antibiotic era when up to 25% of all deaths were attributed to infections. Thus, there is an urgent need for antibiotics that are targeted, easy to produce, and retain potency after repeated use.

To address this need, the inventors propose developing smart RNA antibiotics that are pathogen specific. These drugs will act like a trojan horse, silently moving from microbe to microbe within a consortium of microbes until they enter the targeted pathogen, where they will trigger cell death by inducing intracellular antimicrobial toxins. This feature makes RNA antibiotics highly selective, thereby leaving beneficial bacteria unscathed. Smart RNA will limit the emergence of drug resistance by only affecting targeted pathogens. More broadly, smart RNA represents a next-generation RNA editing technology that is designable and functionally versatile, like CRISPR-Cas technologies that edit DNA, albeit simpler to design and deliver to pathogens in complex microbiomes.

Finally, smart RNA antibiotics are potent and have the potential to be tested against a variety of pathogens in a rapid pipeline (days) development of smart RNA that meets specific patient needs. An automated RNA drug design platform can be created that revolutionizes antibiotic design by enabling the rapid creation of potent, specific, and affordable drugs that support individual patient needs in real time, with development costs that are orders of magnitude below those of chemical therapeutics. To identify methods to deliver DNA-encoding smart RNA, a method can be used that tracks DNA as it enters multiple microbes in a consortium. This uses a cat-RNA that writes barcodes in each microbe taking up DNA within the consortium in parallel. As such, this tool represents a new approach for quickly identifying the best candidate therapeutics, which is complementary to smart RNA antibiotics. Moreover, it has a wider application for other consortia-labeling applications where robust screening is required.

These and other aspects of the disclosure are described in detail below.

### I. Smart RNA

### A. Ribozymes

Ribozymes (ribonucleic acid enzymes) are RNA molecules that have the ability to catalyze specific biochemical reactions, including RNA splicing in gene expression, similar to the action of protein enzymes. The 1982 discovery of ribozymes demonstrated that RNA can be both genetic material (like DNA) and a biological catalyst (like protein enzymes), and contributed to the RNA world hypothesis, which suggests that RNA may have been important in the evolution of prebiotic self-replicating systems. The most common activities of natural or in vitro-evolved ribozymes are the cleavage or ligation of RNA and DNA and peptide bond formation. For example, the smallest ribozyme known (GUGGC-3′) can aminoacylate a GCCU-3′ sequence in the presence of PheAMP. Within the ribosome, ribozymes function as part of the large subunit ribosomal RNA to link amino acids during protein synthesis. They also participate in a variety of RNA processing reactions, including RNA splicing, viral replication, and transfer RNA biosynthesis. Examples of ribozymes include the hammerhead ribozyme, the VS ribozyme, Leadzyme, and the hairpin ribozyme. Attempts have been made to develop ribozymes as therapeutic agents, as enzymes which target defined RNA sequences for cleavage, as biosensors, and for applications in functional genomics and gene discovery.

Despite having only four choices for each monomer unit (nucleotides), compared to 20 amino acid side chains found in proteins, ribozymes have diverse structures and mechanisms. In many cases they are able to mimic the mechanism used by their protein counterparts. For example, in self-cleaving ribozyme RNAs, an in-line SN2 reaction is carried out using the 2′ hydroxyl group as a nucleophile attacking the bridging phosphate and causing 5′ oxygen of the N+1 base to act as a leaving group. In comparison, RNase A, a protein that catalyzes the same reaction, uses a coordinating histidine and lysine to act as a base to attack the phosphate backbone.

Like many protein enzymes, metal binding is also critical to the function of many ribozymes. Often these interactions use both the phosphate backbone and the base of the nucleotide, causing drastic conformational changes. There are two mechanism classes for the cleavage of phosphodiester backbone in the presence of metal. In the first mechanism, the internal 2′-OH group attacks phosphorus center in a SN₂ mechanism. Metal ions promote this reaction by first coordinating the phosphate oxygen and later stablizing the oxyanion. The second mechanism also follows a SN₂ displacement, but the nucleophile comes from water or exogenous hydroxyl groups rather than RNA itself. The smallest ribozyme is UUU, which can promote the cleavage between G and A of the GAAA tetranucleotide via the first mechanism in the presence of Mn²⁺. The reason why this trinucleotide rather than the complementary tetramer catalyzes this reaction may be because the UUU-AAA pairing is the weakest and most flexible trinucleotide among the 64 conformations, which provides the binding site for Mn²⁺.

Phosphoryl transfer can also be catalyzed without metal ions. For example, pancreatic ribonuclease A and hepatitis delta virus (HDV) ribozymes can catalyze the cleavage of RNA backbone through acid-base catalysis without metal ions. Hairpin ribozyme can also catalyze the self-cleavage of RNA without metal ions but the mechanism is still unclear. Ribozymes can also catalyze the formation of a peptide bond between adjacent amino acid by lowering the activation entropy.

Although ribozymes are quite rare in most cells, their roles are sometimes essential to life. For example, the functional part of the ribosome, the biological machine that translates RNA into proteins, is fundamentally a ribozyme, composed of RNA tertiary structural motifs that are often coordinated to metal ions such as Mg²⁺ as cofactors. In a model system, there is no requirement for divalent cations in a five-nucleotide RNA catalyzing trans-phenylalanation of a four-nucleotide substrate with 3 base pairs complementary with the catalyst, where the catalyst/substrate were devised by truncation of the C3 ribozyme.

The best-studied ribozymes are probably those that cut themselves or other RNAs, as in the original discovery by Cech and Altman; however, ribozymes can be designed to catalyze a range of reactions (see below), many of which may occur in life but have not been discovered in cells. RNA may catalyze folding of the pathological protein conformation of a prion in a manner similar to that of a chaperonin.

Since the discovery of ribozymes that exist in living organisms, there has been interest in the study of new synthetic ribozymes made in the laboratory. For example, artificially produced self-cleaving RNAs that have good enzymatic activity have been produced. Self-cleaving RNAs were identified by in vitro selection of RNAs originating from random-sequence RNAs. Some of the synthetic ribozymes that were produced had novel structures, while some were similar to the naturally occurring hammerhead ribozyme. In 2015, researchers engineered a tethered ribosome that works nearly as well as the authentic cellular component that produces all the proteins and enzymes within the cell. The techniques used to create artificial ribozymes involve directed evolution. This approach takes advantage of RNA's dual nature as both a catalyst and an informational polymer, making it easy for an investigator to produce vast populations of RNA catalysts using polymerase enzymes. The ribozymes are mutated by reverse transcribing them with reverse transcriptase into various cDNA and amplified with error-prone PCR. The selection parameters in these experiments often differ. One approach for selecting a ligase ribozyme involves using biotin tags, which are covalently linked to the substrate. If a molecule possesses the desired ligase activity, a streptavidin matrix can be used to recover the active molecules.

Although not true catalysts, the creation of artificial self-cleaving riboswitches, termed aptazymes, has also been an active area of research. Riboswitches are regulatory RNA motifs that change their structure in response to a small molecule ligand to regulate translation. While there are many known natural riboswitches that bind a wide array of metabolites and other small organic molecules, only one ribozyme based on a riboswitch has been described, glmS. Early work in characterizing self-cleaving riboswitches was focused on using theophylline as the ligand. In these studies, an RNA hairpin is formed which blocks the ribosome binding site, thus inhibiting translation. In the presence of the ligand, in these cases theophylline, the regulatory RNA region is cleaved off, allowing the ribosome to bind and translate the target gene. Much of this RNA engineering work was based on rational design and previously determined RNA structures rather than directed evolution as in the above examples. More recent work has broadened the ligands used in ribozyme riboswitches to include thymine pyrophosphate. Fluorescence-activated cell sorting has also been used to engineering aptazymes.

Ribozymes have been proposed and developed for the treatment of disease through gene therapy. One major challenge of using RNA based enzymes as a therapeutic is the short half-life of the catalytic RNA molecules in the body. To combat this, the 2′ position on the ribose is modified to improve RNA stability. One area of ribozyme gene therapy has been the inhibition of RNA-based viruses. A type of synthetic ribozyme directed against HIV RNA called gene shears has been developed and has entered clinical testing for HIV infection. Similarly, ribozymes have been designed to target the hepatitis C virus RNA, SARS coronavirus (SARS-COV), Adenovirus and influenza A and B virus RNA. The ribozyme is able to cleave the conserved regions of the virus's genome which has been shown to reduce the virus in mammalian cell culture. Despite these efforts by researchers, these projects have remained in the preclinical stage.

### B. Group I Introns

Group I introns are large self-splicing ribozymes. They catalyze their own excision from mRNA, tRNA, and rRNA precursors in a wide range of organisms. The core secondary structure consists of nine paired regions (P1-P9). These fold to essentially two domains—the P4-P6 domain (formed from the stacking of P5, P4, P6 and P6a helices) and the P3-P9 domain (formed from the P8, P3, P7 and P9 helices). The secondary structure mark-up for this family represents only this conserved core. Group I introns often have long open reading frames inserted in loop regions.

Splicing of group I introns is processed by two sequential transesterification reactions. The exogenous guanosine or guanosine nucleotide (exoG) first docks onto the active G-binding site located in P7, and its 3′-OH is aligned to attack the phosphodiester bond at the 5′ splice site located in P1, resulting in a free 3′-OH group at the upstream exon and the exoG being attached to the 5′ end of the intron. Then the terminal G (omega G) of the intron swaps the exoG and occupies the G-binding site to organize the second ester-transfer reaction: the 3′-OH group of the upstream exon in P1 is aligned to attack the 3′ splice site in P10, leading to the ligation of the adjacent upstream and downstream exons and release of the catalytic intron. Two-metal-ion mechanism seen in protein polymerases and phosphatases was proposed to be used by group I and group II introns to process the phosphoryl transfer reactions, which was unambiguously proven by a high-resolution structure of the Azoarcus group I intron in 2006.

C. “smart RNA”

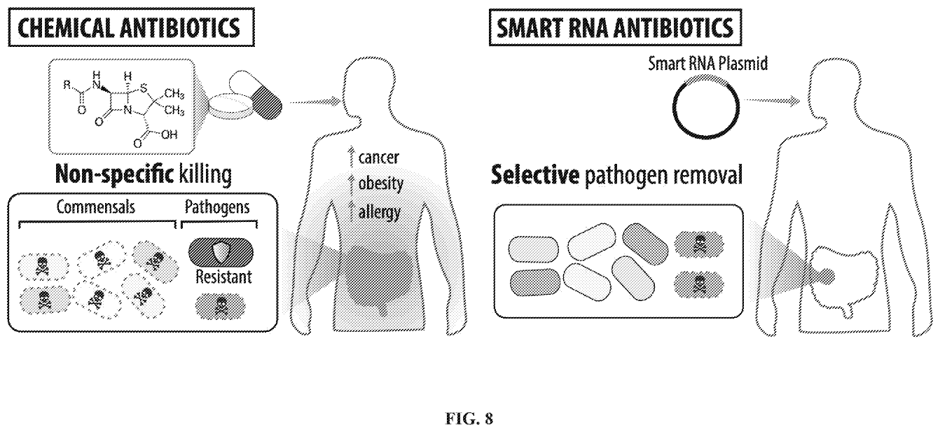

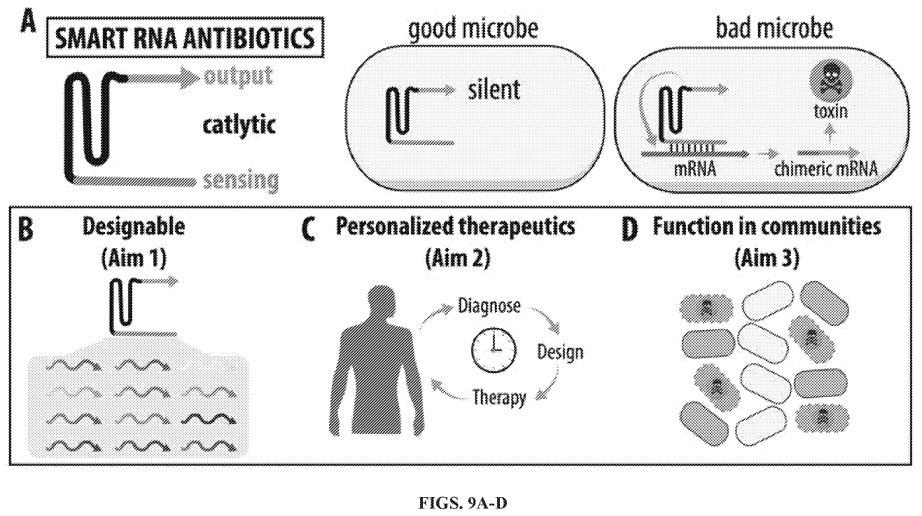

The present disclosure is directed as utilizing Group I ribozymes to generate a new class of custom antibiotics that address current unmet needs. Smart RNA antibiotics (FIG. 8) that are built using a versatile RNA-editing tool whose activity is dependent upon cellular information (mRNA sequences). These drugs are built by assembling three modular RNA parts (FIG. 9A), including (i) an output module that encodes a protein, (ii) a sensing or input module that activates protein synthesis when it encounters a pathogen-specific mRNA, and (iii) an RNA-editing catalytic module that uses information from the sensing module to control when and where toxin production occurs. These drugs are safe because they are non-toxic for the host and microbiome, and smart because they are only active and toxic against the targeted pathogen.

By varying each module, smart RNA antibiotics can easily be created that kill pathogens via a diversity of mechanisms. Different pathogen-specific mRNA can be used to trigger toxin synthesis (FIG. 9B), and a plethora of protein toxins can be coded as outputs. This modular design makes smart RNA fast and affordable to develop for any pathogen using a patient's microbiome profile, which can be used to inform the best designs to achieve potency and specificity while minimizing the potential for drug-resistance evolution. As the RNA editing module is compact, RNA drugs can be easily packaged within DNA and delivered to diverse pathogens within microbiomes at desired doses using existing gene transfer mechanisms (FIG. 9D).

The core hypothesis is that RNA antibiotics will enable a new paradigm for personalized medicine (FIG. 9C), where doctors sequence a patient's microbiome at the infection site to identify species that are good (commensal), bad (pathogenic), or potentially bad (pathobionts). The inventors posit that this kind of patient data can be used to rapidly (days) design and create customized RNA drugs that are tailored to each patient's needs. Antibiotics are some of the most common drugs used in hospitals and represent a considerable healthcare cost. Since the DNA used to code for smart RNA can be synthesized cheaply and on demand, the inventors hypothesize that these antibiotics represent an affordable new drug option with much needed therapeutic activities.

Biocontainment is another area where smart RNA constructs may be employed. While synthetic cells can be useful to various organisms, such as plants, it would be valuable to have such biotechnologies be conditional such that they only survive in certain settings. Smart RNA could be engineered to restrict synthetic cells and therefore affect biocontainment. For example, an engineered microbe may have a smart RNA antibiotic as a feature. One could couple synthesis to loss/gain of a signal that is related to movement from the controlled environment to a non-controlled environment, then that killing would occur when the non-controlled environment was entered. As a specific example, the inventors propose coupling rhizosphere microbial consortia fitness to the presence of a plant, such that plant removal causes the engineered microbial consortium to die. The same concept can be applied to a sequence of a microbiome in soil or plant rhizosphere where the smart RNA kills those cells based on proximity to a plant (close it lives; distant it dies). It could also be applied using proximity to any environment or microbiome containing cells, such as gut, soil, wastewater, and built environments.

### II. Pathogens and Anti-Pathogen Proteins

An infection is the invasion of tissues by pathogens, their multiplication, and the reaction of host tissues to the infectious agent and the toxins they produce. An infectious disease, also known as a transmissible disease or communicable disease, is an illness resulting from an infection. Infections can be caused by a wide range of pathogens, most prominently bacteria and viruses. Hosts can fight infections using their immune system. Mammalian hosts react to infections with an innate response, often involving inflammation, followed by an adaptive response. Infectious diseases resulted in 9.2 million deaths in 2013 (about 17% of all deaths).

In accordance with the present disclosure, smart RNA may be designed to target virtually any pathogen that carries a unique DNA sequence encoding an mRNA, as mRNA are ubiquitous biomolecules required for life. The pathogen can be a bacterium, a fungus or a virus. The anti-pathogen protein can be an enzyme, a toxin, an inhibitor, an RNA-guided endonuclease (e.g., a CRISPR-Cas system), antimicrobial peptides and proteins, toxins, nucleases, and more. It can also be variants of these biomolecules that have been subjected to rational design or laboratory evolution to create functional variants with new protein sequences. Examples of each of discussed below.

### A. Pathogens

Bacterial pathogens include Escherichia coli (ETEC, EPEC, EAEC), Lactobacillus iners, Clostridioides difficile, Lactobacillus plantarum, Lactobacillus johnsonii, Lactobacillus gasseri, Lactobacillusparagasseri, Lactobacillus iners, Lactobacillus crispatus, Bacillus anthracis, Clostridium botulinum, Francisella tularensis, Yersinia pestis, Staphylococcus aureus, Salmonella typhimurium, Streptococcus pneumoniae, Vibrio cholerae, Shigella sonnei, Pseudomonas aeruginosa, Neisseria gonorrhoeae, Mycobacterium tuberculosis, Legionella pneumophilia, Heliobacter pylori, Chlamydia trachomatis, Cornybacterium diptherieae, Acinetobacter baumannii, Enterococcus Faecalis, and Campylobacter jejuni.

Fungal pathogens include Aspergillus, Blastomyces, Coccidioides, Cyrptococcus, Paracoccidioides, Histoplasma, Candida, Talaromyces, Pneumocystis, and Sporothrix.

Candida species are important human pathogens that are best known for causing opportunist infections in immunocompromised hosts (e.g., transplant patients, AIDS sufferers, and cancer patients). Infections are difficult to treat and can be very serious. Aspergillus can and does cause disease in three major ways: through the production of mycotoxins, through induction of allergenic responses, and through localized or systemic infections. With the latter two categories, the immune status of the host is pivotal. The most common pathogenic species are Aspergillus fumigatus and Aspergillus flavus.

Cryptococcus neoformans can cause a severe form of meningitis and meningo-encephalitis in patients with HIV infection and AIDS. The majority of Cryptococcus species live in the soil and do not cause disease in humans. Cryptococcus laurentii and Cryptococcus albidus have been known to occasionally cause moderate-to-severe disease in human patients with compromised immunity. Cryptococcus gattii is endemic to tropical parts of the continent of Africa and Australia and can cause disease in non-immunocompromised people. Other Basidiomycota species such as Malassezia furfur and Trichosporon asahii can cause opportunistic infections in immunosuppressed patients or catheterized individuals.

Histoplasma capsulatum can cause histoplasmosis in humans, dogs and cats. Pneumocystis jirovecii (or Pneumocystis carinii) can cause a form of pneumonia in people with weakened immune systems, such as premature children, elderly individuals, transplant patients and AIDS patients. Stachybotrys chartarum or “black mold” can cause respiratory damage and severe headaches. It frequently occurs in houses in regions that are chronically damp. Subcutaneous zygomycosis caused by Basidiobolus ranarum is a rare disease and affects mainly children and men. Conidiobolus coronatus can infect both humans and animals. Blastomycosis is a rare but serious invasive infection caused by Blastomyces dermatitidis that primarily affects the lungs. Paracoccidioidomycosis is a systemic fungal disease caused by members of the genus Paracoccidioides. Coccidioidomycosis is a fungal disease caused by Coccidioides immitis or Coccidioides posadasii, which are frequently responsible for community-acquired pneumonia in areas where it is endemic. Talaromyces marneffei is the causative agent of talaromycosis which typically affects immunocompromised individuals. Members of the genus Fusarium can cause infections of the nail and the cornea in healthy individuals but can cause disseminated infection in immunocompromised individuals. Members of the order Mucorales, particularly those belonging to the genera Rhizopus and Mucor are responsible for mucormycosis, which can be life-threatening in immunocompromised individuals. Encephalitozoon cuniculi is an obligate intracellular fungus that can cause disease in immunocompromised individuals.

Viral pathogens include viral pathogens that represent important health concerns. These pathogens include respiratory viruses such as Adenoviruses, Avian influenza, Influenza virus type A, Influenza virus type B, Measles, Parainfluenza virus, Respiratory syncytial virus (RSV), Rhinoviruses, SARS-COV, MERS-COV and SARS-COV-2, gastro-enteric viruses such as Coxsackie viruses, enteroviruses such as Poliovirus and Rotavirus, hepatitis viruses such as Hepatitis B virus, Hepatitis C virus, Bovine viral diarrhea virus (surrogate), herpesviruses such as Herpes simplex 1, Herpes simplex 2, Human cytomegalovirus, and Varicella zoster virus, retroviruses such as Human immunodeficiency virus 1 (HIV-1), and Human immunodeficiency virus 2 (HIV-2), as well as Dengue virus, Hantavirus, Hemorrhagic fever viruses, Lymphocytic choromeningitis virus, Smallpox virus, Ebola virus, Rabies virus, West Nile virus and Yellow fever virus. Some non-limiting viral antigens include hepatitis B virus HBV surface and core antigens, influenza virus haemagglutinin and neuroaminidase antigens, West Nile virus envelope protein (E) and premembrane protein (prM), Dengue virus 80E subunit protein, Ebola virus glycoprotein, HIV envelope protein gp41 and gp120, or fragments thereof.

The pathogen can also be a parasite. These include African Sleeping Sickness (African trypanosomiasis), Amebiasis (Entamoeba histolytica Infection), American Trypanosomiasis (Chagas Disease), Ancylostomiasis (Hookworm), Angiostrongyliasis (Angiostrongylus Infection), Ascariasis (Ascaris Infection, Intestinal Roundworms), Cryptosporidiosis (Cryptosporidium Infection), Cutaneous Larva Migrans (CLM, Ancylostomiasis, Hookworm), Dirofilariasis (Dirofilaria Infection), Echinococcosis (Cystic, Alveolar Hydatid Disease), Elephantiasis (Filariasis, Lymphatic Filariasis), Entamoeba hartmanni, Entamoeba histolytica, Infection (Amebiasis), Enterobiasis (Pinworm Infection), Giardiasis (Giardia Infection), Kala-azar (Leishmaniasis, Leishmania Infection), Liver Flukes (Clonorchiasis, Opisthorchiasis, Fascioliasis), Malaria (Plasmodium Infection), Microsporidiosis (Microsporidia Infection), Schistosomiasis (Bilharzia), Toxoplasmosis (Toxoplasma Infection), Trichinellosis (Trichinosis), Trichinosis (Trichinellosis), Trichomoniasis (Trichomonas Infection), and Whipworm Infection (Trichuriasis, Trichuris Infection).

### B. Anti-Pathogen Proteins

Anti-pathogen proteins include a vast array of natural and synthetic peptide and protein sequences. This includes type I toxins (e.g., CcdB, Hok Fst, TisB), type II toxins (e.g., ccdB, ParE, MazF, YafO), type III toxins (e.g., ToxN), type IV toxins (e.g., DarT), type V toxins (e.g., GhoT), type VI (e.g., SocB), type VII (e.g., TglT), restriction enzymes (e.g., Dpn1, EcoRV, EcoR1, BamHI, BsbI), Cas proteins (e.g., Cas3, Cas9, Cas10), exonucleases (e.g., exonuclease IX, exonuclease X), antimicrobial peptides (e.g., nisin, cecropins, defensins, interfron-I), antifungal peptides (e.g., aurein 12, aflatoxin), antiviral peptides (e.g., defensins, gramicidin D, caerin 1, maximin 3, Epi-1), antiparasitic peptides (e.g., Epi-1, Jellein), and signaling inhibitors (e.g., two-component inhibitors such as RWJ-49815, quorum-sensing inhibitors such as lactonase).

Toxins (Type 1 to VII) are derived from toxin-antitoxin systems that are found across different prokaryotes where they are associated with a range of different functions. When encoded on plasmids, their role is typically to ensure correct plasmid inheritance. CcdB is a well-characterized example that kills cells by inhibiting the GyrA subunit of DNA gyrase, which induces breaks in DNA and cell death. Toxins can also be found in genomes and play a role in genome stabilization.

Antimicrobial peptides (AMPs) are a large class of biomolecules with broad-spectrum antimicrobial activities. They often function through interactions with membranes, for example, nisin inhibits growth by generating pores in cell membranes. Another example, cecropin, induces cell lysis and reduces membrane integrity. In addition, large natural repertoires of these biomolecules synthetic AMPs have been created through rational design and laboratory evolution. Some of these AMPs are broad host range, while others have some cell specificity.

Repurposed CRISPR-Cas systems can also be used as antimicrobials. For example, these RNA-guided endonucleases can be used to induce cleavage within a targeted cell's genome to create double-stranded breaks. If left unresolved, these breaks are lethal and induce cell death. Different CRISPR-Cas systems have been used including S. pyogenes Cas9 (SpCas9).

### III. Cancer Cell Targets and Anti-Cancer Proteins

Cancer is a group of diseases involving abnormal cell growth with the potential to invade or spread to other parts of the body. These contrast with benign tumors, which do not spread. Possible signs and symptoms include a lump, abnormal bleeding, prolonged cough, unexplained weight loss, and a change in bowel movements. While these symptoms may indicate cancer, they can also have other causes. Over 100 types of cancers affect humans and result in millions of deaths each year. Examples of cancers a discussed below.

In accordance with the present disclosure, smart RNA may be designed to target virtually any pathogen that carries a unique DNA sequence encoding an mRNA. The sequences to be delivered by the smart RNA that can inhibit or kill cancer cells are also discussed below.

### A. Cancers

Cancer cells that may be treated with the smart RNAs of the present disclosure include but are not limited to cells from the bladder, blood, bone, bone marrow, brain, breast, colon, esophagus, gastrointestine, gum, head, kidney, liver, lung, nasopharynx, neck, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; Mullerian mixed tumor; nephroblastoma; hepatoblastoma; carcinosarcoma; mesenchymoma, malignant; Brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; Kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### B. Anti-cancer Proteins

One well known type of anti-cancer agent is a tumor suppressor gene (TSG), or anti-oncogene, is a gene that regulates a cell during cell division and replication. If a cell grows uncontrollably, it may result in cancer, and if the cell has a tumor suppressor gene that is mutated, this can result in a loss or reduction in its ability to regulate cell growth/division. In combination with other genetic mutations, this could allow the cell to grow abnormally and become cancerous. The loss of function for TSGs is therefore significant in the development of human cancers, even more so than the activation of so-called oncogenes.

TSGs can be grouped into the following categories: caretaker genes, gatekeeper genes, and more recently landscaper genes. Caretaker genes ensure stability of the genome via DNA repair and subsequently when mutated allow mutations to accumulate. Meanwhile, gatekeeper genes directly regulate cell growth by either inhibiting cell cycle progression or inducing apoptosis. Lastly, landscaper genes regulate growth by contributing to the surrounding environment, when mutated can cause an environment that promotes unregulated proliferation. The classification schemes are evolving as medical advances are being made from fields including molecular biology, genetics, and epigenetics.

Examples include pVHL, APC, CD95, ST5, YPEL3, ST7, ST14, p16, BRCA1, BRCA2, p16, p53, Rb, VHL, APC, NF1, PTCH, PTEN, TUSC2, SWI/SNF, and BCL2.

### IV. Biocontainment

Microbial containment can be achieved using strains whose growth depends on nutrient supplementation (Lopez & Anderson, 2015; Wright et al., 2015) kill switches that link survival to an environmental condition (Chan et al., 2016), and sequence entanglement that prevents gene transfer (Blazejewski et al., 2019). Cells can also be embedded in materials like alginate beads to decrease the risk of escape or genetic exchange with communities (Tang et al., 2021). Among these approaches, the former is required for applications in environmental matrices like soils. However, such genetically encoded approaches remain arduous to use in consortia because each individual killing target requires significant effort to create and optimize. Gene knockouts can be arduous for undomesticated microbes, and kill switches require significant tuning. Also, these approaches are susceptible to mutational escape, though the use of redundancies in biocontainment circuits has shown efficacy (Wright et al., 2015). Currently, there is a need for more versatile containment that can use a range of killing mechanisms, can be built using simple and easy to deliver genetic circuits, can be created with high specificity thereby avoiding off-target effects, can be created as cocktails of antibiotics to target one or more strains in a consortia using different mechanisms, and can be designed in a way that minimizes the evolution of drug resistance. Also, there is a need for technologies that use genome remodeling to shape the environmental niche of a microbe and to create metabolic dependencies on an easy-to-remove member of the ecosystem, e.g., a plant, built material, or gut microbiome.

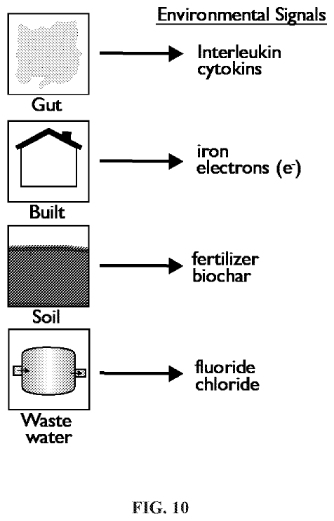

This disclosure describes the next generation of biocontainment measures that can be applied to any environmental consortium. To do this, a smart RNA can be created that is customized to kill a microbe or consortia and the production or inhibition of these antimicrobials coupled to the presence of molecular signals present in the environment. For example, the production of smart RNA can be repressed in response to naturally arising molecular signatures from the environment in which a microbe or consortia is being contained within. If a microbe or consortia member escapes this environment, the molecular signature will no longer be present, and the production of the smart RNA will be turned on and the microbe killed. Example applications include biocontainment within the human gut where engineered microbes or consortia are dynamically sensing native environmental signals (e.g., interleukin, cytokines) to repress a smart RNA inside each microbe that is customized to kill that host cell (FIGS. 10A-C). In another example, biocontainment in built environments could be achieved by conditionally expressing smart RNA in response to environmental signals that include inorganic materials (e.g., iron) or electrons (FIGS. 10A-C).

In another example, the production of smart RNA can be repressed in response to a user-provided additive that serves to contain the microbe or consortia in that environment. This approach also allows for genetically modified organisms to be economically removed at any time through the removal of the molecular signatures, to enable the safe applications of synthetic biology in complex environmental settings. For example, in soil environments microbes and consortia can be programmed to conditionally repress smart RNA in response to soil amendments (e.g., fertilizers, biochar). Likewise, for applications in wastewater treatment additives such as fluoride and chloride could be used (FIGS. 10A-C).

The genetically encoded antibiotics are expected to be extraordinarily versatile because they are easy and quick to design and produce, they can be designed with RNA guides that target one or more species in parallel, they can be multiplexed easily as they are simple genetic programs, and ultimately affordable to develop to meet diverse cell programming needs in real time using computational tools arising from these efforts. The universal RNA memory system, which is compatible with consortia-scale information storage.

In one application, the authors posit that native microbes derived from the rhizosphere will selectively sense a plant signal called an exudate. To identify microbes that sense that signal, the inventors will isolate the community and evaluate which genes are conditional upon the signal. This transcriptomic analysis will identify genes that are differentially expressed in the presence of individual exudates or signals unique to the environmental setting targeted. As a specific example, the inventors propose to identify the genetic control elements (i.e., promoter and transcription factor pairs) responsible for exudate sensing in microbes associated with the plant rhizosphere. These mined elements can then be deployed into consortium members to program conditional antibiotic production. Using the transcriptomics data as a guide, they will use ribozymes to couple conditional promoter activity to the production of an antibiotic, such that cells proximal to a plant making the exudate do not express the antibiotic and those removed from that setting switch on the production of the antibiotic.

The genetically encoded antibiotics are expected to be extraordinarily versatile because they are easy and quick to design and produce, they can be designed with RNA guides that target one or more species in parallel, they can be multiplexed easily as they are simple genetic programs, and ultimately affordable to develop to meet diverse cell programming needs in real time using computational tools arising from these efforts. The universal RNA memory system, which is compatible with consortia-scale information storage.

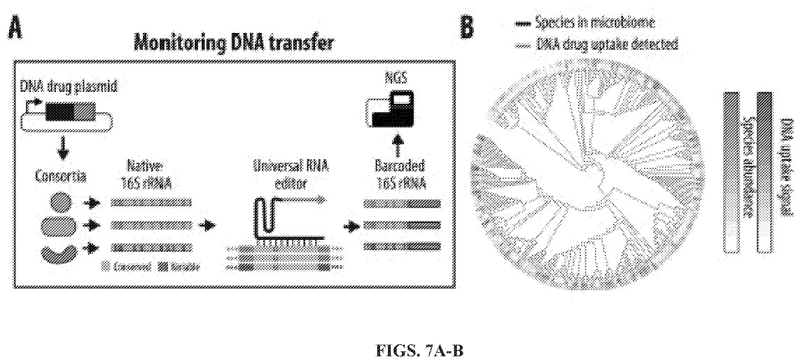

At present there are also no simple strategies to monitor synthetic microbe containment in soils. Also, once cells die, there is still a risk that environmental DNA encoding synthetic circuits could be taken up via gene transfer. Thus, there is a need for simple tools to monitor synthetic cells, synthetic DNA, and gene transfer in soil. One solution for monitoring containment will be to code a consortia-scale information storage device into each engineered consortia member. Also, the inventors will create different information storage devices for each antibiotic to understand their relative performance. This universal RNA memory system (FIG. 7B) will allow tracking who contains this DNA in time and space. This approach will be of relevance to multiple agencies (USDA, FDA, and EPA) that are regulating genetically engineered organisms and biotechnologies. The inventor believe that this technology will have broad utility because it can be diversified to monitor gene flow arising from conjugation, transformation, and transduction.

### V. Signal Production and Biosensing

Living sensor can be used in hard-to-image settings like soils to provide information about chemicals sensed, such as toxins. These sensors couple the detection of an environmental chemical to the production of biomolecules that generate visual outputs that are imaged, changes in the freezing point of water released from the soil, and volatile indicator gases that diffuse out of the soil. With these approaches, complex genetic circuits are required to program cells, which are typically domesticated organisms. Currently, there is a need for simple, versatile reporter systems that can generate a range of chemical signal outputs, can be built using simple and easy to deliver genetic circuits, and can be created to generate biorthogonal signaling channels in real environmental settings like soils.

The inventors posit that native microbes derived from environmental consortia can be genetically programmed to generate conditional signals that report on specific environmental conditions. Specifically, microbes and consortia will naturally respond to different environmental signatures and conditions that result in changes to their transcriptome (i.e., mRNA content of the cell). For example, in the presence of a pollutant certain mRNA within consortia will be activated that could include catabolic genes, stress response genes, or efflux pumps. Smart RNA can be designed to target these conditional mRNA and transduce these signals into different protein outputs that include detectable reporter genes, such as a volatile gas produced in the headspace of a natural material (e.g., soil). Thus, when a specific environmental signature is present, the smart RNA can be used to produce a detectable reporter signal that can be easily detected. This approach for biosensing is facile because conditional mRNA are readily identified using RNA sequencing and smart RNA can be designed to target any mRNA and produce any protein output.

### VI. Producing and Delivering Smart RNAs

Creating smart RNA against a target pathogen begins with identifying the native mRNA to be targeted, the location within this native mRNA to target, and the antimicrobial protein to be used. If the application is to target pathogens, metagenomic sequencing data and bioinformatic analysis can be used to identify mRNA sequences that are unique or highly dissimilar to other sequences in the microbiome. To identify the best site to target within a single or many mRNA, libraries of different smart RNA can be created, and laboratory selections performed. For example, commercial DNA synthesis can be used to create thousands of DNA fragments encoding RNA guide sequences and cloned into smart RNA plasmids. Highly active smart RNA designs can then be identified by transforming these plasmids into the target pathogens, performing laboratory selections whereby the fitness of the host cell is coupled to the activity of the smart RNA, and performing sequencing to identify the designs with the highest activity. Optimal antimicrobial proteins can also be identified by screening different output genes and quantifying the effect on growth and/or survival. The specificity of smart RNAs can be quantified by assessing their effects on the growth of non-target organisms, such as commensals. A complementary approach for creating smart RNA is to leverage computational design algorithms that use microbiome data and computer modeling to predict the best designs.

To deliver smart RNAs to a targeted microbiome a variety of different mechanisms can be used. Natural mechanisms of gene transfer (e.g., conjugation, transduction, transformation, and vesicle transfer) provide one option. For example, to use conjugation, DNA encoding a smart RNA can be added to a conjugative plasmid and inserted into a conjugative donor cell. This donor cell can then be introduced into a microbiome, whereby it will transfer this plasmid across cells in the community. Any natural or synthetic conjugative plasmid may be used (e.g., ColE1 derivatives, F plasmids, pRK2, pRSF1010, pMB1). Transduction can also be used by embedding DNA or RNA encoding smart RNA into a phage particle (e.g., P1 phage, Enterobacteria phage T2, M13 phage, T5 Phage) and introducing this into a microbiome. Beyond natural mechanisms, synthetic mechanisms of gene transfer can be used to introduce DNA, for example, synthetic nanoparticles.

### VII. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Modifying Bacterial Cell Targets

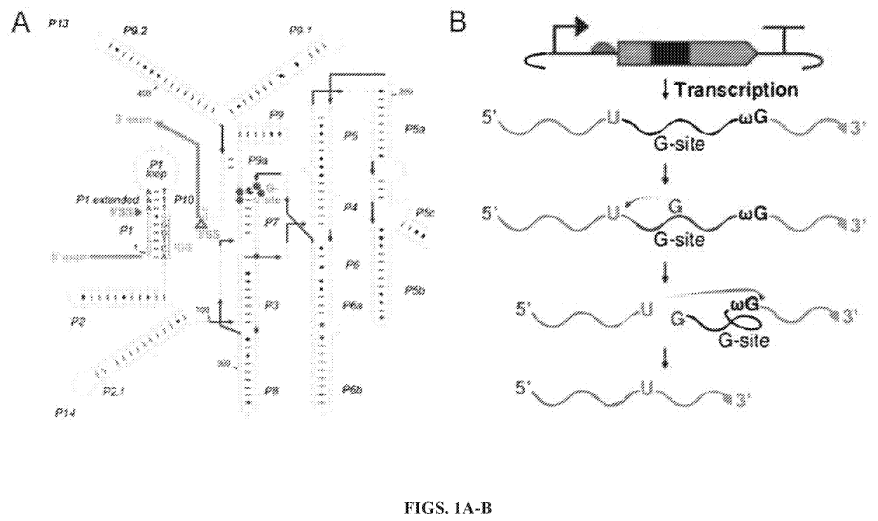

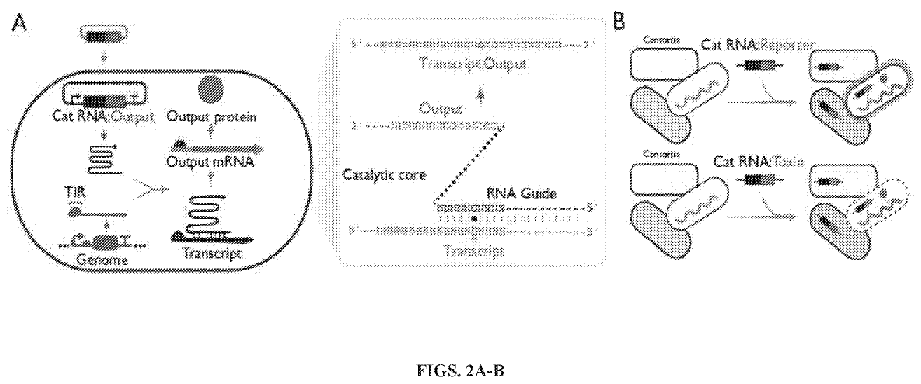

RNA antimicrobials. RNA antimicrobials are based upon an engineered trans-splicing ribozyme referred to herein as cat-RNA, which is derived from the Tetrahymena ribozyme. Cat-RNA is a trans-splicing ribozyme (the catalytic core) that is amended to contain an RNA guide at the 5′ end and an output RNA at the 3′ end (FIG. 1A). The RNA guide can be programmed to hybridize to any host-mRNA sequence and can vary in sequence. Upon hybridization, the catalytic core catalyzes a splicing reaction of the RNA output onto a targeted RNA, which can also have any sequence desired. The one sequence constraint for the splicing reaction (inset FIG. 2A) is that the RNA guide and targeted RNA transcript must contain one conserved uracil.

Ribozyme-regulation of protein expression. While the cat-RNA can be used to target any host RNA imaginable for splicing, host RNA splicing site can be targeted to lead to the production of a hybrid transcript (FIG. 2A) that is competent for protein expression. When the splicing reaction results in the addition of a translation initiation sequence (TIR, also called a ribosome binding site) from a host RNA to the 5′ end of the RNA output, which is an ORF that is not translation competent, it creates a transcript that can translate the protein coded as the RNA output. This approach can be used to regulate the synthesis of any protein imaginable (FIG. 2B), ranging from the production of a toxic protein to induce cell death to a visual reporter protein produced to create detectable signals for diagnostics to a protein that is a component of a more complex synthetic cellular circuit (FIG. 2B) to proteins that improve protein fitness in different cellular environments.

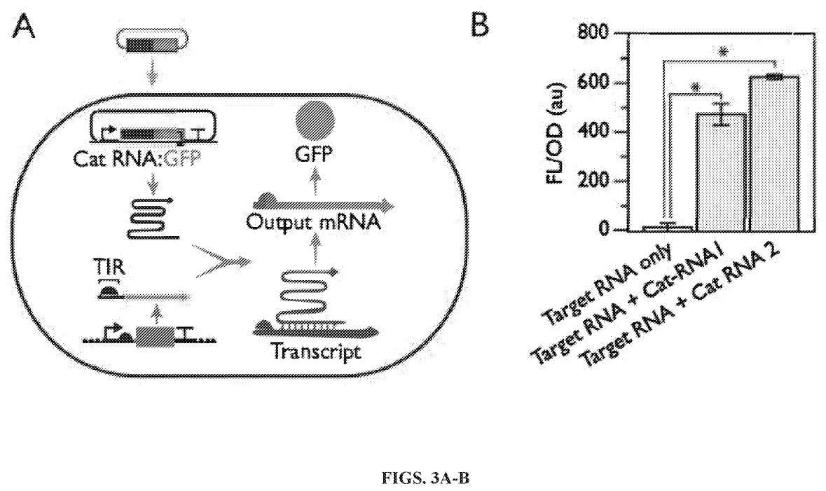

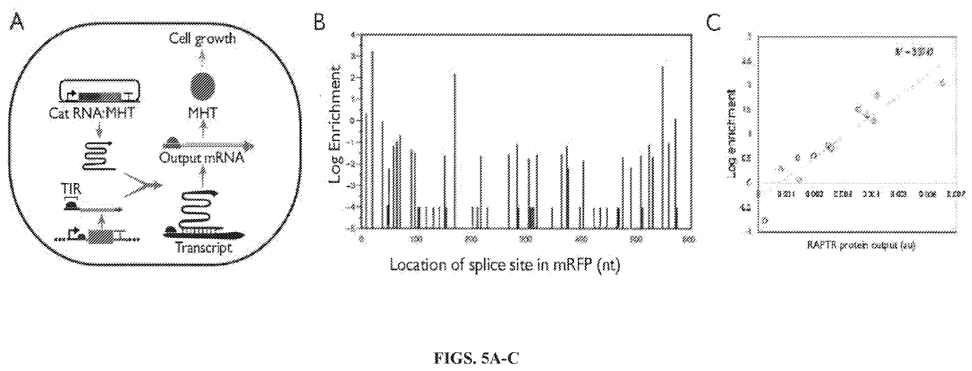

cat-RNA can selectively activate the expression of detectable reporter proteins at high levels using cellular RNA. To demonstrate a proof of principle, the inventors first measured the efficiency of the splicing reaction performed by Cat-RNA in bacteria. To do this, they used an output RNA that encodes a green fluorescent protein (GFP). The target RNA was a non-native mRNA expressed in Escherichia coli (FIG. 3A). Two cat-RNA were designed that contained unique guide RNA designed to read different regions of the target RNA. As a negative control, the inventors used cells that contained the target RNA only but no Cat-RNA. These results showed that when Cat-RNA is present with its target RNA, a significant amount of GFP is produced as measured by fluorescence measurements. The inventors have also performed this measurement with other reporters including a gas-producing methyl halide transferase (MHT) (FIG. 5). Additionally, the inventors have demonstrated that this reaction works in a range of microbes using a slightly different architecture for catalytic RNA, including marine (Vibrio), soil (Pseudomonas), and sediment (Shewanella) microbes (data not shown).

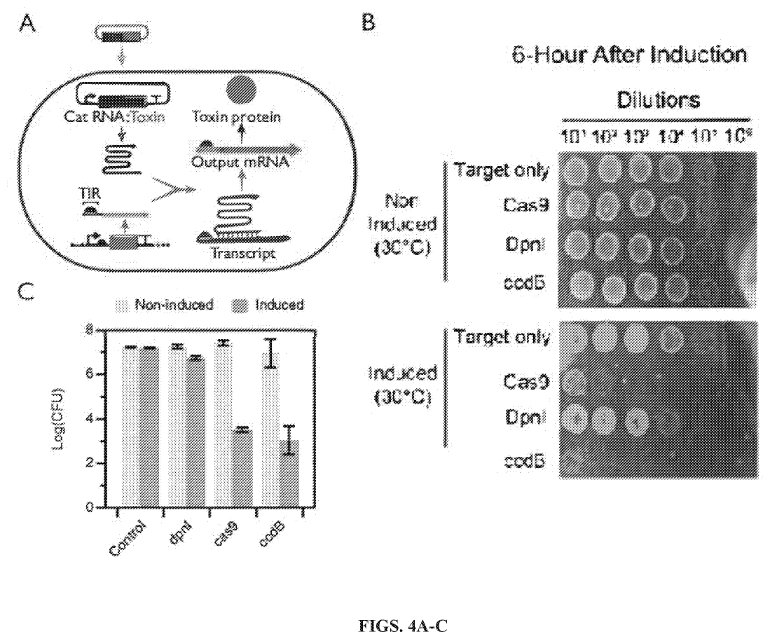

cat-RNA can induce cell death in response to cellular RNA demonstrating that they function as an antibiotic following gene transfer. With an efficient Cat-RNA mechanism in hand, the inventors next sought to confirm that Cat-RNA can use a cellular mRNA to produce antimicrobial proteins in bacteria (FIG. 4A). As a proof of principle, three toxic genes were chosen: (1) a CRISPR-Cas system that cleaves the host's genome when produced to induce cell death, (2) a Dpn1 restriction enzyme that cleaves the host's genome to induce cell death, and (3) a CcdB protein that inhibits a critical cellular enzyme (DNA gyrase) to induce cell death. Cat-RNA built with these outputs were expressed in Escherichia coli from a plasmid that had been transformed into cells. This cat-RNA was under the control of a cumate-inducible promoter, such that cells could be transformed and single colonies could be obtained containing the DNA encoding cat-RNA without cell death occurring.

Single colonies obtained from transforming each plasmid were grown overnight in LB liquid medium and diluted 1 in 30-fold to an approximate optical density at 600 nm of 0.1. Cells were then divided into two groups and grown either in the presence or absence of 60 μM cumate, which induces the transcription of cat-RNA. Cells were grown at 30° C. for 6 hours allowing for the induction of cat-RNA transcription in the cultures containing cumate. Following this incubation, the inventors measured the colony forming units (CFU) by adding different aliquots of the culture to LB agar plates and growing these overnight at 37° C. Multiple death genes have been tested using this approach including an inhibitor of DNA gyrase (CcdB), a restriction endonuclease (Dpn1), and an RNA-guided DNA nuclease (CRISPR Cas). All death genes decreased colony forming units (CFU) relative to a non-induction control in which each cat-RNA is not transcribed. The ccdB output is the most effective and reduces cell number 5-orders of magnitude compared to the negative control. This proves that RNA antimicrobials can use cellular RNA to produce a variety of toxic proteins that induce cell death.

Varying the RNA guide and mRNA targets to understand efficiency. In their current work, the inventors are investigating the relation between RNA guide, target sequence, and splicing efficiency. Specifically, they are testing the activity of many cat-RNA variants that are designed to target different mRNA in E. coli cells. To identify active variants, the inventors are using a growth selection whereby the output from a trans-splicing reaction produces an enzyme, called a methyl halide transferase (MHT), that allows an engineered strain of E. coli to grow in an M63 minimal media without amino acids (FIG. 5A). Thus, protein production increases fitness of cells rather than killing.

To perform these selections, the library of cat-RNA designs is transformed into E. coli cells, and these cells are grown under the growth selection. Cells containing more efficient cat-RNA designs grow faster than cells containing less efficient cat-RNA designs and thus increase in their abundance. Cells are grown over the course of several days. At the end of the experiment, the DNA plasmids in the cell population are then isolated and next-generation sequencing performed. From this, the inventors calculate the relative-fold enrichment of each design after selection by comparing abundance before and after selection. FIG. 5B shows how this approach can be used to rapidly score a wide range of splicing targets on a single mRNA to create cat-RNA with varying activities in regulating protein production. The fold enrichment values are proportional to protein production and thus catalytic RNA trans-splicing activity.

FIG. 5C shows the enrichment of these sequences from a selection experiment compared with the measured MHT activity (protein output) for several variants in individual cells. The latter measurement is directly monitoring the relative activities of each catalytic RNA. This confirms that the-fold enrichment data from the selections shown in FIG. 5A is correlated with protein output levels produced from each variant.

In sum, multiple prototypes have been shown to function in E. coli.These experiments employed three different catalytic RNA have been coded into a mobile DNA element (plasmid), which produce three different protein toxins upon reaction with a targeted RNA transcript, including a DNA gyrase (CcdB), a restriction endonuclease (Dpn1), and an RNA-guided DNA nuclease (CRISPR Cas). Following a short incubation, these RNA antibiotics killed 99.99%, 99.90% and 90.00% of the cells, respectively. Additionally, the inventors have shown that multiple sites in a single RNA target in a cell can be targeted by varying the RNA guide in the cat-RNA and yield spliced RNA molecules that produce protein, albeit at varying levels. While these experiments have shown the versatility of splicing reactions that yield protein production (specifically, leading to the production of a methyl halide transferase), they are being used to make a protein that improves the fitness of E. coli rather than killing. These designs will ultimately be used to control the production of toxins and improve antimicrobial efficacy.

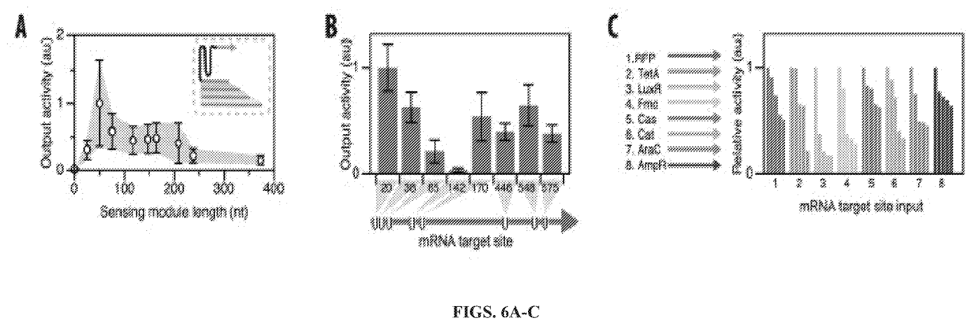

Diverse mRNA can trigger smart RNA output production. In smart RNA, the sensing module can be varied to direct binding to different locations within any native mRNA, provided that it contains an uracil (Su et al., 2021). Smart RNA can then use those different binding sites to trigger the production of a protein toxin. The inventors have shown that mRNA motifs with a range of lengths (25 to 200 nucleotides) can be used as a trigger (FIG. 6A). To see if smart RNA output levels vary when different sites are targeted in the same mRNA, the inventors compared a series of designs where the sensing module was 50 nucleotides long (FIG. 6B). A majority yielded a detectable signal, although they varied in the output magnitude. To see if different mRNA molecules in a cell can be used to trigger a smart RNA output, the inventors evaluated the performance of >10³ designs that target eight different mRNA (FIG. 6C). These designs presented a detectable output compared to the original prototype. Taken together, these results show that the sensing module can be rapidly diversified to make smart RNA conditional upon different cellular mRNA signatures of varying lengths.

The inventors have also shown that different RNA in a cell can be targeted to trigger the production of the spliced RNA molecules. To date, they have demonstrated activation of protein production by having the cat-RNA target a wide range of transcripts including transcripts coding for a lactamase, dioxygenase, fluorescent proteins, transcription factors, and other enzymes. These have been mRNA produced from genes encoded on plasmids and the host genome. These have been mRNA produced from native and heterologous genes. While these experiments have shown the versatility of splicing reactions across different RNA targets, they are being used to make a protein that improves the fitness of E. coli rather than killing.

Robust consortia programming using a new strategy for gene transfer. To program microbes in environmental consortia, there is a need to efficiently deliver DNA to taxonomically diverse microbes with unique functions. To identify which delivery approach is best, e.g., rhizosphere consortia or soil containing microbes enriched with specific phenotypes, one must compare the efficiencies of different strategies, which is arduous. To simplify this process, the inventors have used an RNA editing tool that writes barcodes onto conserved genetic motifs found across all bacteria. When DNA encoding this tool enters a microbe, it writes these barcodes adjacent to sequences that are commonly used to identify species present in microbiomes. They have shown that this tool allows for rapid identification of microbes in a 200-member sludge microbiome that takes up synthetic DNA by conjugation, i.e., ~50% of the consortia members (FIG. 7B). This tool represents a new innovation that the inventors plan to employ to rapidly screen the best vehicles for delivering DNA to microbiomes and the retention of that DNA as communities grow and change. This approach is compatible with studies across hard-to-image environmental microbiomes, e.g., those in soils, with divergent abiotic and biotic properties.

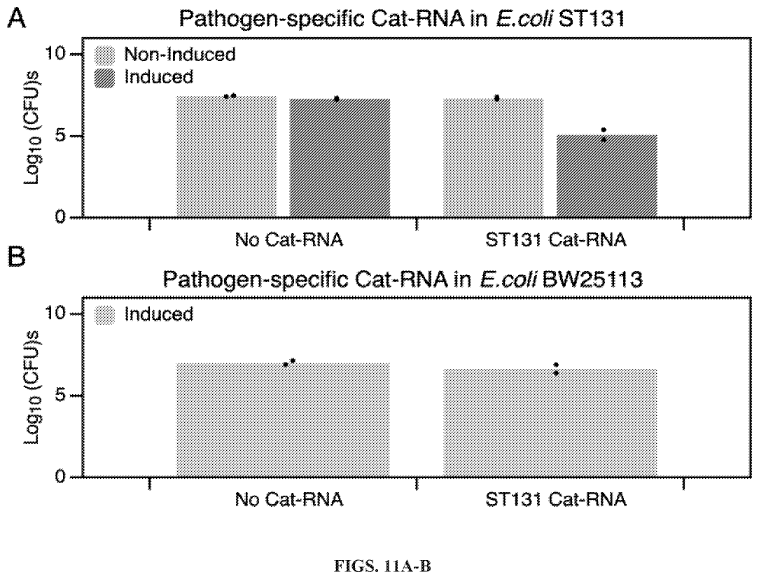

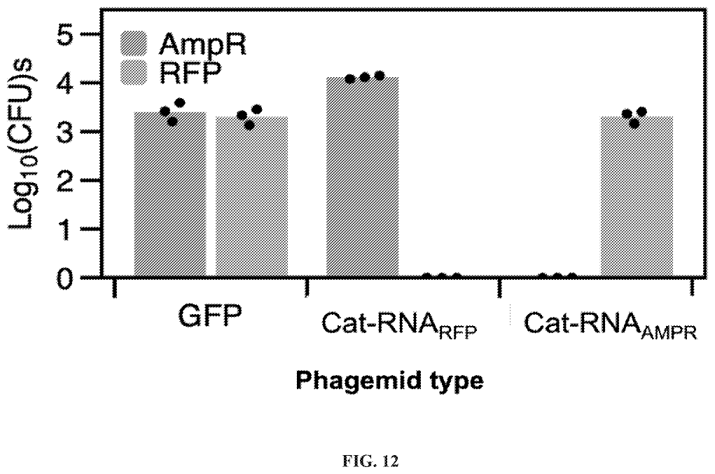

Cat-RNA can selectively kill a pathogenic E. coli by targeting pathogen-specific mRNA. Importantly, the inventors can design Cat-RNA to selectively activate a toxin in response to pathogenic-specific mRNA signatures. In this way, Cat-RNA would remain silent if inside a non-pathogen (i.e., a commensal) but be selectively activated when present in a pathogen. FIGS. 11A-B shows a series of experiments in which (i) the inventors demonstrate that they can selectively kill a pathogen called ST131 by targeting a pathogen-specific mRNA called aaaC3 and (ii) that these designs do not affect a non-pathogenic strain of E. coli lacking the targeted mRNA.

Cat-RNA can be delivered to cells using a non-replicative bacteriophage called phagemid. A key claim of the present disclosure is that one can deliver Cat-RNA using diverse mechanisms such as conjugation, transformation, and transduction. In this result, the inventors show that they can use a non-replicative bacteriophage P1 (a bacterial virus) called a phagemid to deliver DNA encoding the inventors' Cat-RNA. This is an important proof of principle because the delivery of genetically encoded antibiotics is a major hurdle of these technologies, and this data shows a viable option for delivery is through transduction using bacteriophage. See FIG. 12.

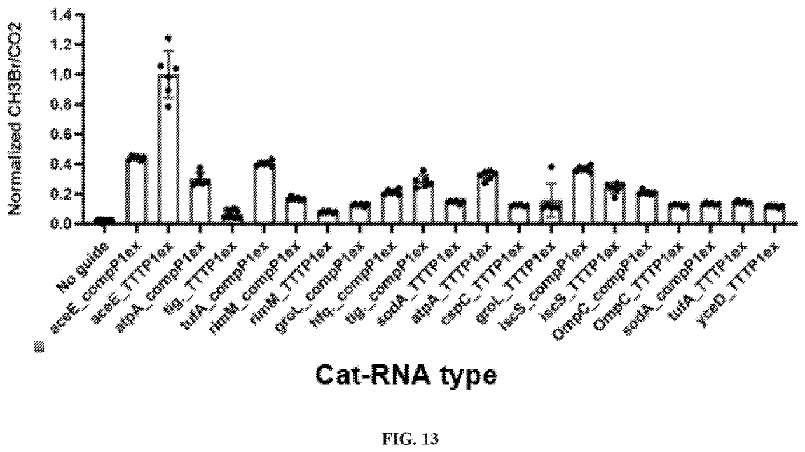

Cat-RNA can use low-copy genome-encoded genes to activate the production of protein outputs. A key claim of the disclosure is that one can use diverse mRNA to activate the cat-RNA. This data (FIG. 13) provides examples of cat-RNA activating the expression of a reporter protein through many different endogenous genes encoded on the genome of E. coli.This supports the claim that endogenous genes can be used as targets for Cat-RNA.

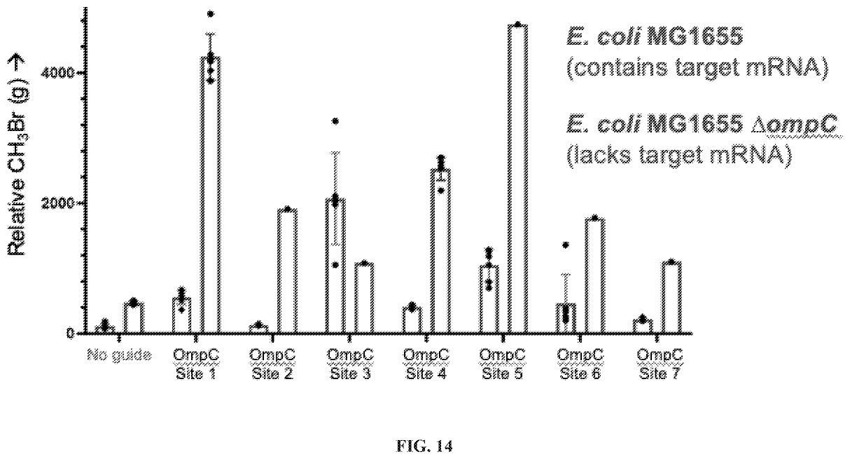

A second piece of data (FIG. 14) also supports this same claim. In this experiment, the inventors are focusing on several designs of Cat-RNA that target an endogenous gene called OmpC. This experiment includes an additional control in which cells lacking the OmpC (−OmpC) are compared to cells that have the OmpC (+OmpC) on the genome. While some designs show the Cat-RNA is active when the target mRNA is not present (i.e., they are not specific), the inventors see many instances where the cat-RNA is off without the target mRNA present.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
Ayre et al., Proc. Natl. Acad. Sci. 96, 3507-3512 (1999).
Blazejewski et al., Science 365, 595-598 (2019).
Carter et al., BMC Mol. Biol. 11, 84(2010 ).
Chan et al., Nat Chem Biol 12, 82-86 (2016).
Kwon et al. Mol. Ther. 12, 824-834 (2005).
Lan et al., Science 280, 1593-1596 (1998).
Lopez & Anderson, ACS Synth. Biol. 4, 1279-1286 (2015).
Phylactou et al., Nat. Genet. 18, 378-381 (1998).
Ryu et al., Mol. Ther. 7, 386-395 (2003).
Su et al., Nature 596, 603-607 (2021).
Sullenger & Cech, Nature 371, 619-622 (1994).
Wright et al., ACS Synth. Biol. 4, 307-316 (2015).
Tang et al., Nat Chem Biol 17, 724-731 (2021).

1. A ribonucleic acid (RNA) segment comprising a catalytically functional core from a trans-splicing catalytic RNA, a synthetic guide RNA (sgRNA), and an open reading frame (ORF) encoding a peptide or protein that encodes a protein or peptide of interest, wherein the sgRNA targets a RNA transcript such that a trans-splicing reaction by said catalytic RNA results in the introduction of said ORF into said RNA transcript such that a translational control signal of said RNA transcript is positioned in operable relation to said ORF.

2. The RNA segment of claim 1, wherein said RNA segment lacks any translational control signals and/or lacks an origin of replication.

3. The RNA segment of claim 1, wherein said ORF encodes a detectable marker, a protein conferring growth/survival capability on said cell, a protein that generates overt information, a protein that produces a signal containing information, an antimicrobial, an anti-fungal agent, an anti-viral agent, anti-archaean agent or a toxin.

4. The RNA segment of claim 1, wherein said translational control signal is a translation initiation signal and/or ribosome binding site.

5. The RNA segment of claim 1, wherein said catalytic RNA is derived from Tetrahymena, such as a group I intron, or is synthetic

6. The RNA segment of claim 1, wherein said sgRNA:
(a) targets a eukaryotic, mammalian, human, bacterial, fungal, archaean or viral transcript;
(b) is fully or partially complementary to said RNA transcript; and/or
(c) comprise all natural ribonucleotides, all non-natural ribonucleotides, or a mix of natural and non-natural ribonucleotides.

7. The RNA segment of claim 1, wherein said catalytically functional core is about 200, 300 or 400 ribonucleotides in length, is about 200-500, 300-500, or 400-500 ribonucleotides in length, is about 200-600, 300-600, or 400-600. ribonucleotides in length, is about 200-800, 300-800 or 400-800 ribonucleotides in length, or is about 200-100-, 300-1000 or 400-1000 ribonucleotides in length.

8. The RNA segment of claim 1, wherein said sgRNA is about 25 to about 375 ribonucleotides in length.

9. The RNA segment of claim 1, wherein said RNA transcript is a native transcript, such as a transcript arising from a low copy number gene.

10. The RNA segment of claim 9, wherein the RNA transcript is an engineered transcript, such as one comprising a non-hydrolyzable nucleic acid backbone.

11. A nucleic acid vector encoding the RNA segment of claim 1.

12. The vector of claim 11, wherein said vector is an RNA or DNA vectors, such as a viral vector, such as a baculovirus vector or a phage.

13. The vector of claim 11, wherein said vector is DNA vector, such as a non-viral vector, such as a plasmid (e.g., self-mobilizing or non-self-mobilizing).

14. A pharmaceutical formulation comprising the RNA segment of claim 1.

15. A cell comprising the RNA segment of claim 1.

16. A method of expressing a protein or peptide in a cell comprising contacting said cell with an RNA segment of claim 1 under conditions permitting uptake of said RNA segment or vector by said cell and trans-splicing of said RNA transcript by said catalytic RNA.

17. The method of claim 16, wherein said cell is a bacterial cell, a fungal cell, a parasite cell, a mammalian cell, or a human cell.

18. The method of claim 16, wherein said RNA segment is contacted with said cell as naked RNA.

19. The method of claim 16, wherein said RNA segment is contacted with said cell packaged in a nanoparticle or liposome.

20. The method of claim 16, wherein said RNA segment encodes a peptide or protein that is toxic to said cell, and said method results in the death of said cell.

21. The method of claim 16, wherein said cell is part of a consortium of cells.

22. The method of claim 21, wherein said consortium contains one or more organisms that are pathogenic to a plant, insect, mammal or human.

23. The method of claim 16, wherein said RNA segment encodes a peptide or protein that it confers a growth or survival advantage to said cell under all or selective environments.

24. The method of claim 23, wherein said cell is part of a consortium of cells.

25. The method of claim 24, wherein said consortium is beneficial to a plant, insect, mammal or human.

26. The method of claim 24, wherein said consortium expresses a protein of interest.

27. The method of claim 16, wherein contacting results in transduction of, transformation of or conjugation with said cell.

28. The method of claim 16, wherein said ORF encodes a detectable marker and said method further comprises detecting expression of said detectable marker, such as quantitative detection.

29. The method of claim 16, wherein trans-splicing results in constitutive expression of said peptide or protein.

30. The method of claim 16, wherein trans-splicing permits conditional expression of said peptide or protein when other conditions are met.

31. A method of biocontained supplementation comprising:
(a) providing an engineered organism that provides a benefit to a plant or microbe and comprises a ribonucleic acid (RNA) segment comprising a catalytically functional core from a trans-splicing catalytic RNA, a synthetic guide RNA (sgRNA), and an open reading frame (ORF) encoding a peptide or protein that encodes a protein or peptide that is toxic to said engineered organism, wherein the sgRNA targets an RNA transcript such that a trans-splicing reaction by said catalytic RNA results in the introduction of said ORF into said RNA transcript such that a translational control signal of said RNA transcript is positioned in operable relation to said ORF, and wherein the RNA segment is expressed conditionally when said engineered organism is outside a defined environment; and
(b) contacting said engineered organism with said plant or microbe.

32. A method of biocontained supplementation comprising:
(a) providing an engineered organism that encodes a gene product that provides a benefit to a plant or microbe and comprises a ribonucleic acid (RNA) segment comprising a catalytically functional core from a trans-splicing catalytic RNA, a synthetic guide RNA (sgRNA), wherein the sgRNA targets an RNA transcript that encodes said gene such that a trans-splicing reaction by said catalytic RNA results in the disruption of the RNA transcript encoding said gene product that translation of said RNA transcript is disrupted, and wherein the RNA segment is expressed conditionally when said engineered organism is outside a defined environment; and
(b) contacting said engineered organism with said plant or microbe.

33. A method of biocontained supplementation comprising:
(a) providing an engineered organism that comprises a ribonucleic acid (RNA) segment comprising a catalytically functional core from a trans-splicing catalytic RNA, a synthetic guide RNA (sgRNA), and an open reading frame (ORF) encoding a peptide or protein that encodes a protein or peptide that confers a benefit on a plant or microbe, wherein the sgRNA targets an RNA transcript such that a trans-splicing reaction by said catalytic RNA results in the introduction of said ORF into said RNA transcript such that a translational control signal of said RNA transcript is positioned in operable relation to said ORF, and wherein the RNA segment is expressed conditionally when said engineered organism is inside a defined environment; and
(b) contacting said engineered organism with said plant or microbe.
